(19) United States
(12) Patent Application Publication
(10) Pub. No.: US 2004/0002632 A1
(43) Pub. Date: Jan. 1, 2004

(54) Compliant suction surgical device

(76) Inventors: Christina D'Arrigo, Morris Plains, NJ (US); Daniel Gordon, Newtown, PA (US); John Matonick, Warren, NJ (US); David Stoloff, Whitehouse Station, NJ (US); Rohit Tandon, Belle Meade, NJ (US); Steven Zung-Hong Wu, Hillsborough, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/289,576

(22) Filed: Nov. 7, 2002

Related U.S. Application Data
(63) Continuation-in-part of application No. 10/185,393, filed on Jun. 28, 2002.

(51) Int. Cl. A61B 1/32

(52) U.S. Cl. 600/210

(57) ABSTRACT

A suction device includes a suction cup having a vacuum inlet for connection to a vacuum source. The suction cup has a wall defining a cavity that is in fluid communication with the vacuum inlet and an engagement surface for engaging a tissue surface. The suction cup is formed of a material that minimizes injury to the tissue surface contacted by the engagement surface and tissue captured when the vacuum is applied and permits the captured tissue to be perfused during use of the suction device.

### RELATED APPLICATION

This application is a continuation-in-part from U.S. patent application Ser. No. 10/185,393, filed on Jun. 28, 2002, the entire disclosure of which is incorporated by reference.

### BACKGROUND OF THE INVENTION

1. Field of the Invention

The present invention relates generally to surgical devices, and more particularly, to a suction device for surgical applications in which an organ is supported by the suction device for improved access to the organ.

2. Prior Art

Suction positioning devices for lifting, rotating, and/or supporting an organ are known in the art. They are used in surgical procedures to maintain an organ in a position other than the organ's native position during surgery and are often attached to a surgical retractor, which is used to gain access to the organ through an opening formed in the patient's body. Such suction devices are particularly suited for cardiac bypass surgery, on a beating or stopped heart.

While the suction devices of the prior art have advantages, the vacuum used to support the organ can be excessive and cause tissue damage due to the high vacuum felt by the tissue exposed to the vacuum. For example, high vacuum pressure tends to pull the suctioned tissue against the rim of the suction cup, and the resulting contact or suction force causes local cellular constriction and a reduction in blood flow to the tissue captured within the rim of the cup and the tissue adjacent the captured tissue. Depending upon the length of time the tissue is exposed to the vacuum pressure, suction stabilization devices can induce local tissue damage by preventing normal perfusion of the tissue. Where suction devices are used on the heart, such damage is characterized by epicardial ecchymosis, the long-term clinical effects of which are not known.

During vacuum, mitogenetic factors or “waste product”, which may include free radicals, is produced and trapped in the tissue captured by the vacuum. Upon vacuum or contact release, bloodflow is reestablished to affected areas, thereby releasing the waste products, which in turn can cause damage to the surrounding viable vasculature and myocardium. The more prolonged the exposure of the affected tissue to the vacuum force, the more waste products are generated, thereby increasing the likelihood of reperfusion injury to the surrounding tissue.

U.S. patent application Publication No. 2002/91300 (the “91300 publication”), assigned to Guidant, describes prior art suction cups used to manipulate the heart into a position for the purpose of performing a surgical procedure. The 91300 publication describes suction cups that include a conforming seal attached to the bottom periphery of the cup. The conforming seal is used to form a seal with the heart, while also preventing the heart tissue from being sucked substantially into the internal area of the cup. When the conforming seal of the cup contacts the heart surface and seals against it, the inner volume of air within the cup is evacuated causing the cup to exert a vacuum force on the heart surface. The user may then position the cup to a position other than the heart's native position by using the vacuum force provided by the cup to resist the gravitational force of the heart on the cup.

The flexibility of the cups described in the 91300 publication is not defined. The embodiments described in the 91300 publication rely upon the conforming seal, described as a biocompatible foam, to have the required flexibility to conform to the heart surface to produce the vacuum seal. A prior art suction positioning device sold by Guidant as the Xpose Access Device and determined that the durometer of the cup material was between 45-50 Shore A. The thickness of the wall of the cupped portion was 0.072 inches.

### SUMMARY OF THE INVENTION

Therefore it is an object of the present invention to provide a suction device for surgical procedures that overcomes the problems associated with the prior art.

Accordingly, a self-regulating suction device for applying vacuum to a tissue surface is provided. The suction device includes a suction cup having a vacuum inlet, the suction cup having a wall defining a cavity in fluid communication with the vacuum inlet and an engagement surface for engaging the tissue surface, the suction cup formed of a material that minimizes injury to the tissue surface contacted by the engagement surface due to the applied vacuum by minimizing the tissue surface area exposed to a high vacuum pressure.

### BRIEF DESCRIPTION OF THE DRAWINGS

These and other features, aspects, and advantages of the apparatus and methods of the present invention will become better understood with regard to the following description, appended claims, and accompanying drawings where:

FIG. 1 illustrates a plan view of a surgical retractor device having a suction device mounted thereon.

FIG. 2 illustrates a perspective view of the surgical retractor device of FIG. 1 shown in use for opening the chest wall to provide access to the heart, the suction device shown supporting the heart for improved access thereto.

FIG. 3a illustrates an isometric view of a preferred implementation of a suction device for use with the surgical retractor device of FIG. 1, only the side rail of the surgical retractor being shown therein for clarity.

FIG. 3b illustrates a side view of the suction device and side rail of FIG. 3a.

FIG. 4 illustrates a perspective view of a first preferred implementation of a suction cup of the present invention.

FIG. 5 illustrates a bottom view for the suction cup of FIG. 4 as taken along view 5-5 of FIG. 4.

FIG. 5A illustrates a bottom view of a second embodiment of the suction cup of FIG. 4 as taken along view 5-5 of FIG. 4.

FIG. 6 illustrates a sectional view of the suction cup of FIG. 5 as taken along view 6-6 of FIG. 4.

FIG. 6A illustrates a sectional view of a second embodiment of the suction cup of FIG. 5A as taken along view 6-6 of FIG. 4.

FIG. 7 illustrates an alternative version of a top portion of the suction cup of FIG. 4, the alternative version having a venting valve integrally formed therein.

FIG. 8a illustrates a sectional view of the vacuum inlet portion of FIG. 7 as taken along line 8-8 of FIG. 7, the vacuum inlet portion being shown with the venting valve in the closed position.

FIG. 8b illustrates a sectional view of the vacuum inlet portion of FIG. 7 as taken along line 8-8 of FIG. 7, the vacuum inlet portion being shown with the venting valve in the open (vented) position.

FIG. 9 illustrates an alternative version of the suction cup of FIG. 6, the alternative version having a mesh material inserted in the suction cup portion of the suction cup.

FIG. 10 illustrates a perspective view of the mesh material prior to insertion in the suction cup portion.

FIG. 11 illustrates a side view of yet another alternative version of the suction cup, the alternative version having a closed cell ring disposed on a lower rim of the suction cup portion of the suction cup.

FIG. 12 illustrates a sectional view of the suction cup of FIG. 11 as taken along line 12-12 of FIG. 11.

FIG. 13 illustrates a plan view of a side rail having a mounting means indicated in phantom lines.

FIG. 14 illustrates a sectional view of the side rail and mounting means of FIG. 13 as taken along line 14-14 of FIG. 13.

FIGS. 15a and 15b illustrate sectional views of an alternative version of a mounting means, FIG. 15a showing the mounting means before being secured to the side rail and FIG. 15b showing the mounting means after being secured to the side rail.

FIGS. 16a and 16b illustrate sectional views of another alternative version of a mounting means, FIG. 16a showing the mounting means secured to a first side rail and FIG. 16b showing the mounting means secured to a second side rail having a greater width than the first side rail.

FIGS. 17a and 17b illustrate sectional views of yet another alternative version of a mounting means, FIG. 17a showing the mounting means secured to a first side rail and FIG. 17b showing the mounting means secured to a second side rail having a greater width than the first side rail.

FIGS. 18 and 19 illustrate embodiments of the arm of the suction device.

FIG. 20 depicts comparative pressure distribution within the cupped portions of two suction devices having different durometers.

### DETAILED DESCRIPTION OF THE PREFERRED EMBODIMENT

Although this invention is applicable to numerous and various types of organs and surgical procedures, it has been found particularly useful in the environment of surgical procedures on the heart. Therefore, without limiting the applicability of the invention to surgical procedures on the heart, the invention will be described in such environment.

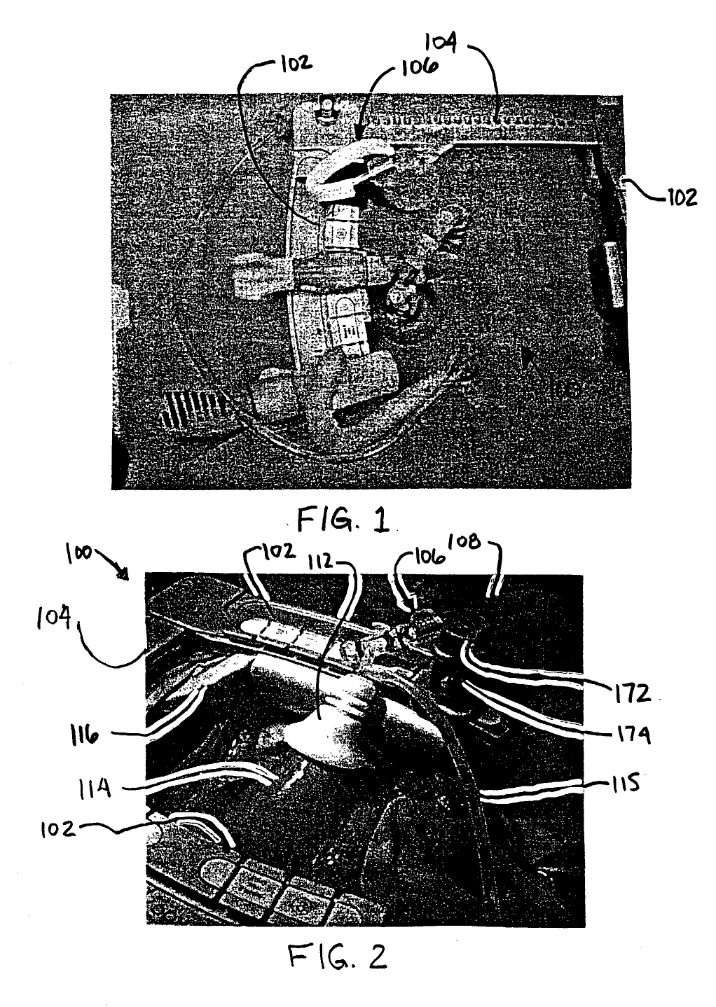

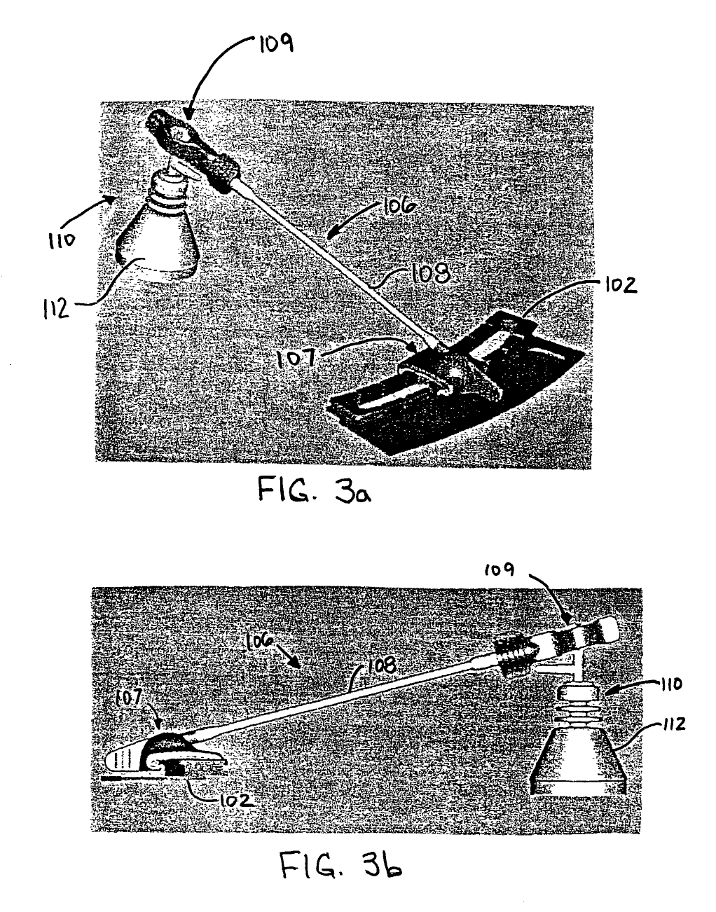

Referring now to FIGS. 1 and 2, there is illustrated a surgical retractor, generally referred to by reference numeral 100. The surgical retractor 100 is useful for retracting the skin to expose a body cavity and/or organ (alternatively referred to herein as “tissue”) for performing a surgical procedure thereon. The surgical retractor generally has one or more attachment members for attachment of accessories, such as a suction device. The attachment members are preferably two side rails 102. The surgical retractor also has at least one transverse rail 104 upon which at least one of the side rails 102 is movable. One and preferably both side rails 102 have means, described fully below for holding accessories useful for the particular surgical procedure being performed. One such accessory is a suction device 106, which is useful for supporting an organ, such as the heart (shown in FIG. 3) during the surgical procedure to provide improved access to the organ and/or body cavity.

Referring now to FIGS. 3a and 3b, the suction device 106 typically has a means 107 for movably engaging the side rail, an arm 108, and a suction cup 110. The arm 108 is movable, preferably by being bendable, and typically cantilevers the suction cup 110 away from the side rail 102. The arm 108 is used to position the suction cup 110 over the organ, after which a suction cup portion 112 engages the organ with an applied vacuum to support the organ in a desired position. A ball joint 109 is provided to allow the suction cup portion 112 to rotate freely into any desired position. The arm 108 is further preferably rotatably disposed relative to the suction cup 110.

In a typical surgical procedure involving the heart 114, after the chest wall 116 is opened, the surgical retractor 100 is placed in the opening with the side rail(s) 102 engaging the opening. The side rails 102 are then slid on the transverse rail 104 to expand the size of the opening. The mounting means 107 is positioned on the side rail 102 and locked thereon to position the suction device 106 such that it will not be an obstruction to the surgical procedure. A vacuum is applied to the suction cup portion 112 by a vacuum source (not shown) and tubing 115. The arm 108 is positioned such that the suction cup portion 112 engages the heart 114 and applies the vacuum to a surface thereof, such as the apical region of the heart. The arm 108 is then raised to partially lift the heart 114 from the chest cavity and support it in the lifted position. In surgical retractors of the prior art, it is required for the arm 108 to be locked in position to support the heart 114. However, as will be discussed below, the suction device 106 of the present invention does not require the arm 108 to be locked.

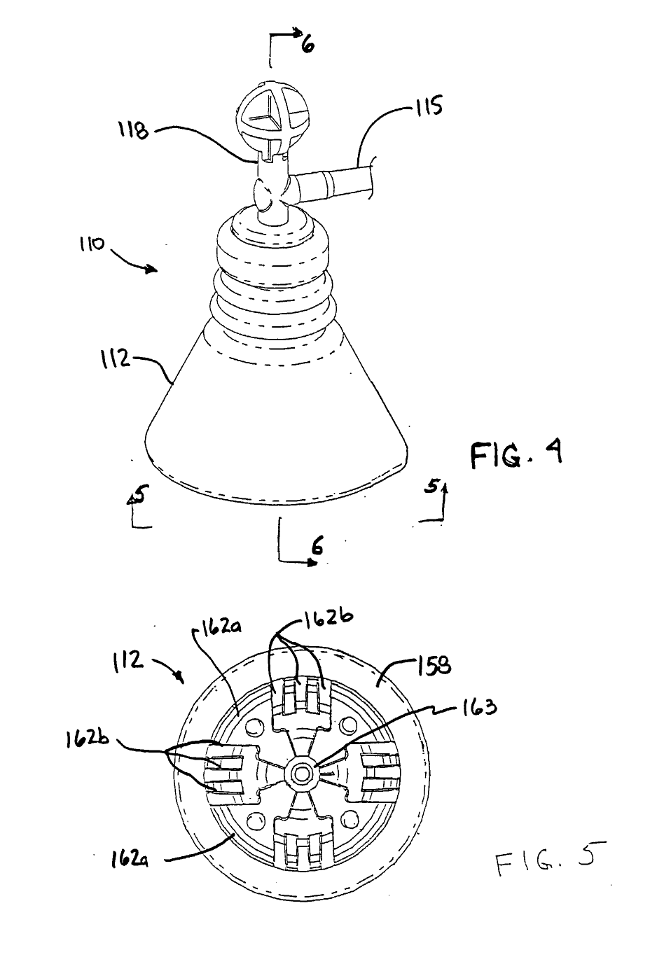

Referring now to FIGS. 4-6, there is shown a preferred implementation of the suction cup 110 of the present invention. Although the suction cup 110 can be of single piece construction, it preferably comprises a suction cup portion 112 and a vacuum inlet portion 118. The suction cup portion 112 is preferably fabricated from a flexible material such as an elastomer. The elastomer is preferably c-flex. The flexible suction cup portion material allows the heart to contract and torque, which allows the heart to maintain its hemodynamic equilibrium.

The vacuum inlet portion 118 is preferably a rigid or semi-rigid thermoplastic. The vacuum inlet portion 118 has a vacuum fitting 120, such as a hose barb, for connection to the vacuum tubing 115. The vacuum fitting 120 has a radial bore 122, which is in fluid communication with an axial bore 124. The vacuum inlet portion 118 further has a ball 126 at an end thereof. The ball 126 is rotatably disposed in a distal adapter 128 connected to a distal end of the arm 108 to form the pivot joint 109.

The suction cup portion 112 is disposed on the vacuum inlet portion 118. The suction cup portion 112 has a wall 130 which defines a cavity 132 which is in fluid communication with the axial bore 126. The vacuum inlet portion 118 and suction cup portion 112 can be fixed together in any manner known in the art. Preferably, the vacuum inlet portion 118 is provided with a channel 134 at an end thereof and the suction cup portion 112 is provided with a corresponding lip 136 which mates with and is fixedly retained in the channel 134.

Referring now to FIGS. 7, 8a, and 8b, there is shown an alternative configuration of the vacuum inlet portion, referred to by reference numeral 118a and in which like numerals denote like features. Vacuum inlet portion 118a differs from vacuum inlet portion 118 in that it has an integral vacuum valve 138 disposed therein. In the alternatively configured vacuum inlet portion 118a illustrated in FIGS. 7, 8a, and 8b, the axial bore, referred to by reference numeral 124a extends throughout the axial length of the vacuum inlet portion 118a. The vacuum inlet portion 118a further has a vent hole 140 in fluid communication with the axial bore 124a. A plunger 142 is slidingly disposed in the axial bore 124a. The plunger 142 has a button 144 on one end thereof for actuating the valve 138 and a seat 146 on another end. A spring 148 is further disposed in the axial bore 124a for biasing the plunger 142 in a closed position illustrated in FIG. 8a in which the button 144 fully extends from the ball 126 and the seat 126 is seated and sealed against a corresponding surface 150 of the axial bore 124a. In the closed position, a vacuum applied to the radial bore 122 is in fluid communication with the cavity 132 of the suction cup portion 112 which can be applied to a surface of the tissue or organ to be supported. When the button 144 is depressed, the valve 138 is switched to an open position, as is illustrated in FIG. 8b. In the open position, the seat 146 is no longer seated against the corresponding surface 150 of the axial bore 124a and the vent hole 140 vents the vacuum applied to the axial bore 124a and cavity 132 to thereby turn the vacuum applied to the tissue or organ off.

Those skilled in the art will appreciate that the valve 138 allows a surgeon to control the applied vacuum independently with his thumb or fingertips and further allows the surgeon to place the suction cup portion 112 at the region where he/she desires and to turn the applied vacuum on or off at will without the need for an assistant. The vacuum valve 138 can be placed at the suction device itself with a button 144 control, as shown, or in another convenient area which permits the surgeon to easily operate the valve without the need for an assistant.

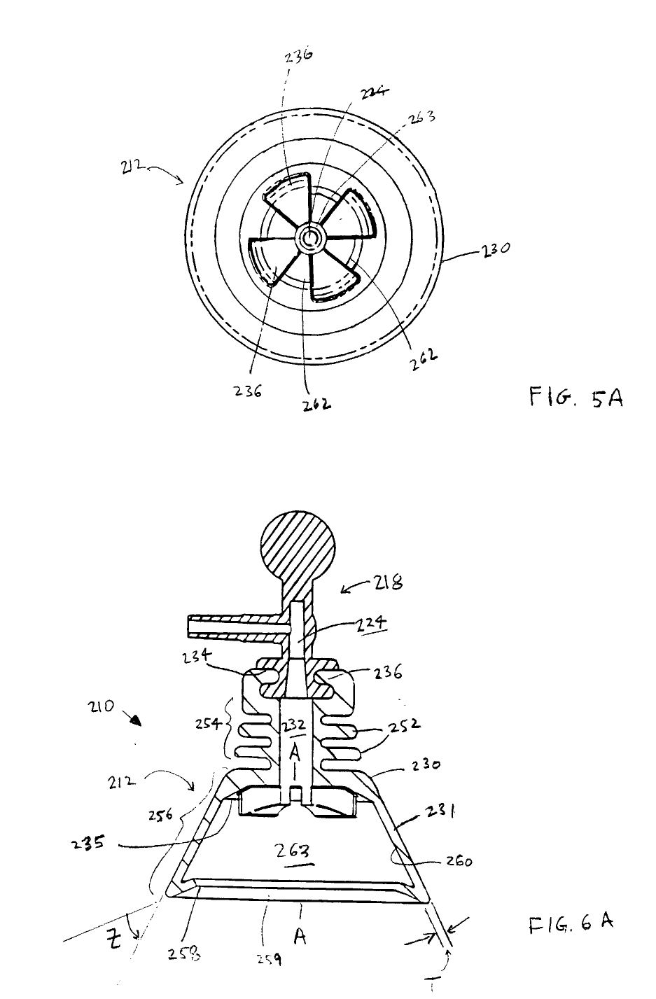

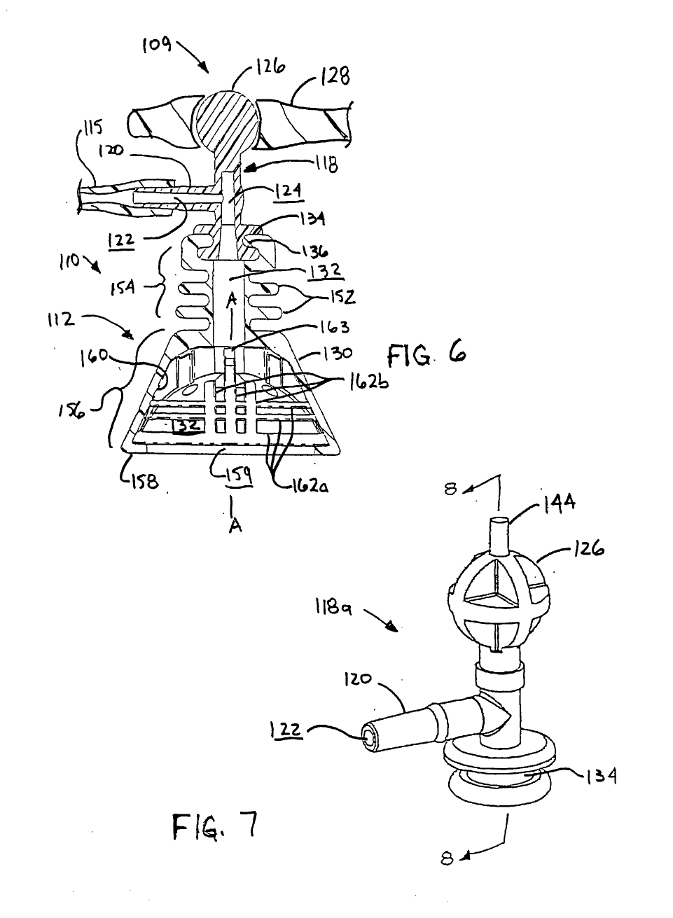

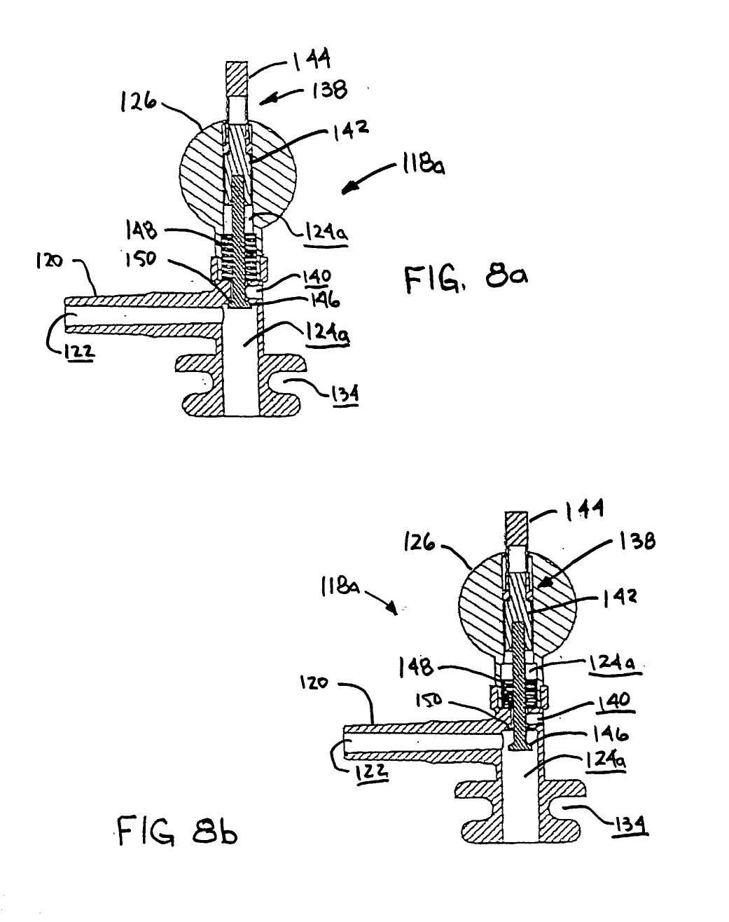

Referring back to FIGS. 5 and 6, there is shown a preferred implementation of the suction cup portion 112 of the suction cup 110. The wall 130 of the suction cup portion 112 preferably has a plurality of ribs 152 for adding flexibility to a neck portion 154 of the suction cup portion 112. The suction cup portion 112 further has a cupped portion 156 which flares outwardly from a central axis A from the neck portion 154 towards a lower rim 158. The lower rim 158 inverts towards the central axis A and defines an opening 159 into the cavity 132. The lower rim 158 further provides a sealing surface that engages against the organ/tissue.

An inner surface 160 of the cupped portion 156 preferably has a plurality of channels 162a, 162b formed thereon. The channels 162a, 162b are more preferably formed in both a circumferential and axial direction, the circumferential channels being referred to by reference numeral 162a and the axial channels being referred to by reference numeral 162b. Both the circumferential and axial channels 162a, 162b are formed at predetermined spacings along the inner surface 160 of the wall 130. Preferably, the axial channels 162 are interconnected at a common point, such as recessed portion 163. The circumferential and axial channels 162a, 162b allow the vacuum to be distributed evenly over the cupped cardiac region and also prevents the possibility of a vacuum line blockage.

Referring now to FIGS. 5A and 6A, an alternative configuration of the suction cup is shown, wherein like numerals denote like features. Suction cup 210 can be of single piece construction, but preferably suction cup 210 comprises a vacuum inlet portion 218 and a suction cup portion 212 that is connected to vacuum inlet portion 218. Vacuum inlet portion 218 and suction cup portion 212 can be fixed together in any manner known in the art. Preferably, vacuum inlet portion 218 is provided with a channel 234 at an end thereof and suction cup portion 212 is provided with a corresponding lip 236 that mates with and is fixedly retained in channel 234.

Suction cup portion 212 includes a neck 254 and a cupped portion 256 that flares outwardly from a central axis A from the neck 254 toward a lower rim 258. The lower rim 258 inverts towards the central axis A and defines an opening 259 into a space 263 that communicates with a cavity 232. The lower rim 258 provides a sealing surface that engages against the organ or tissue. Neck 254 and cupped portion 256 are preferably formed by wall 230. Wall 230 preferably has a plurality of ribs 252 for adding flexibility to neck 254 of suction cup portion 212. Wall 230 defines cavity 232, which is in fluid communication with an axial bore 224 formed in vacuum inlet portion 218.

Wall 230 also includes a side wall 231 having a flexible inner surface 260 that may form a seal with the organ or tissue surface. Unlike wall 130 of prior-described embodiments, side wall 231 does not have a plurality of channels 162a, 162b formed thereon and the attendant additional material required to form channels 162a, 162b. Instead, inner surface 260 of side wall 231 is smooth so as to assist in creating a transient vacuum gradient profile when a vacuum is applied. Wall 230 also includes an upper wall 235 from which shoulders 236 extend downwardly toward lower rim 258. Shoulders 236 form channels 262 between adjacent shoulders 236 and upper wall 235. Channels 262 prevent tissue from blocking cavity 232. Shoulders 236 have a low profile, and preferably extend downward from upper wall 235 about 0.15 to 0.25 inches, most preferably approximately 0.2 inches.

In a preferred embodiment, suction cup 210 is configured to have a controlled leak at the surface that contacts the tissue. In this way, the user can remove the cup without tugging on the heart tissue. Further, a controlled leak permits the vacuum to evacuate space 263 and cavity 232 at a rate that permits the side wall 231 to collapse toward the heart tissue rather than pulling the heart tissue to the side wall 231. The preferred control leak is approximately between 3 and 5 mm Hg/minute, but most preferably about 4 mm Hg/minute.

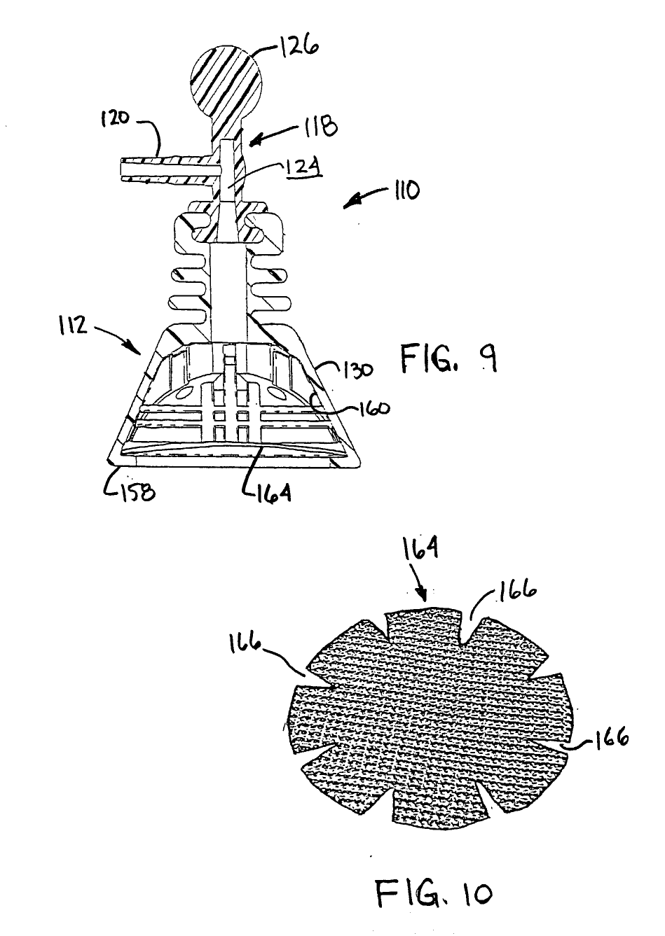

The controlled leak can be accomplished in several ways. A porous foam cushion can be attached to lower rim 258 in the same manner as the closed cell foam 168 depicted in FIGS. 11 and 12 to engage the organ or tissue to be supported. To attain the appropriate controlled leak, the size of the open cells or density of the foam can be varied. Further, the cushion or lower rim surface can be varied such that an uneven seal is attained between the cushion or lower rim. For example, the foam cushion can have channels cut into the foam to permit a controlled leak.

Alternatively, the taper of the cup lip can be varied to provide the controlled leak. In a preferred embodiment, lower rim 258 is tapered to conform with the heart surface, typically the apex of the heart. The angle of lower rim 258 may taper at an angle Z that ranges between 45 degrees and 31 degrees, but most preferably is 38 degrees. The angle of the taper and the cup material may be varied to achieve the preferred controlled leak.

To permit inner wall 260 of cupped portion 256 to flex and conform to the heart surface, cupped portion 256 is preferably fabricated from a compliant material. The compliant material may be an elastomer, having a durometer of less than about 40 Shore A, but preferably between 20 and 40 Shore A. Most preferably the material is C-flex (R70-116-000) having a durometer of 30 Shore A. The thickness T of side wall 231 preferably ranges between 0.054 and 0.066 inches, but most preferably is 0.060 inches. The material permits the apex of the heart to remain substantially undistorted. As a result, the heart is permitted to contract and torque in a way similar to that when it is in its native position, thereby maintaining its hemodynamic equilibrium when the heart is maintained in the desired position.

The suction device works as follows. When the lip 258 of the cupped portion 256 contacts the heart surface and a vacuum is applied to the suction device 210, the inner volume of air within space 263 begins to be evacuated. The rate at which space 263 evacuates is slowed by the controlled leak rate, which as described above, is preferably between approximately 3 and 5 mm Hg/minute. At a given vacuum pressure, a lower portion of the wall 231 collapses against the heart and conforms to the heart, rather than the heart being pulled toward the wall 231 and the heart distorted to conform to the wall.

Experiments were carried out to compare the pressure gradients of an inventive suction device described herein against a prior art cup, the Xpose device. Each device was connected to a vacuum source that provided 250 mm Hg vacuum to the devices, and the devices were placed in contact with the apex of a pig's heart. Probes were inserted at spaced apart vertical locations into the walls of the cupped portions of the devices. FIG. 20 depicts the measured pressures at those positions within the space 263 of the devices. While FIG. 20 depicts distinct areas of internal vacuum pressures within the cups, in fact the pressure gradient is a continuous line, in the case of the inventive device (shown on the right side of FIG. 20), that starts at 35 mm Hg at the lower lip of the cup, indicated as A, increases to 40 mm Hg at a point B, further increases to 110 mm Hg at a point C, and finally is measured as 230 mm Hg at a point D. The pressure above point D ultimately reaches the 250 mm Hg level provided by the vacuum source. This pressure distribution demonstrates that the wall of the inventive device collapses against the heart at point A at 35 mm Hg. Once the apex tissue contacts the wall of the cupped portion, the area below the contact point is cut off from the vacuum source, and the pressure remains at 35 mm Hg. At that point, the vacuum source continues to evacuate the space 263, and when the pressure reaches 40 mm Hg at point B, that portion of wall 231 collapses against the heart tissue and is maintained in that collapsed position by the 40 mm Hg vacuum. Continuing vertically, when the vacuum pressure reaches 110 mm Hg at point C, the portion of wall 231 at point C collapses against the heart tissue. Finally, when the vacuum pressure reaches 230 mm Hg at point D, that portion of the wall collapses against the heart tissue. As one moves vertically from point A to point D, the vacuum pressure required to collapse wall 231 increases due to the structure of the cupped portion. The tissue captured by the inventive device only is exposed to a vacuum pressure of greater than 110 m Hg at point C.

The pressure gradient within the cupped portion of the Xpose device (shown on the left side of FIG. 20) demonstrates that heart tissue captured within the Xpose device is subjected to a pressure of greater than 110 mm Hg at the lip of the cupped portion. Upon application of the 250 mm Hg vacuum pressure, the Xpose cup, which had a measured durometer of approximately 45 to 50 Shore A, did not comply as well to the heart surface. The Xpose cup had a pressure gradient that ranged from 114 mm Hg at a point E at the lip, to 139 mm Hg at a point F, to 169 mm Hg at a point G, and finally to 245 mm Hg at a point H below the upper surface of the cupped portion. Thus, the Xpose device did not comply as well to the heart tissue as the inventive device. The Xpose device did not show any appearance of conforming to the heart tissue. Rather the heart tissue captured within the Xpose device was observed to be distorted and drawn against the cup wall. As a result of this lack of compliance, the space within the Xpose cup, and thus the tissue captured within the cup, was exposed to at least 114 mm Hg vacuum at all locations.

Mean intraluminal pressure within arteries generally ranges from 80 to 90 mm Hg. The peak intraluminal pressure in a typical person is about 120 mm Hg. Any vacuum pressure greater than 120 mm Hg will have the effect of collapsing the arteries/capillaries exposed to the pressure, independent of the cardiac cycle. A vacuum pressure greater than the mean of between 80 and 90 mm Hg deprives the cardiac tissue of blood for a large portion of the cardiac cycle.

It is desirable to supply the cardiac tissue captured by the suction device with blood during the time the heart is positioned by the device. The inventive device performs that function as the majority of the tissue captured by the device is subjected to relatively low vacuum pressures. At least one-third of the volume of the cupped portion of the inventive cup had pressure gradient below 40 mm Hg. In this way, less tissue is exposed to a high vacuum pressure thereby reducing ecchymosis and reperfusion injury. In contrast, all of the heart tissue captured within the Xpose device is subjected to a pressure of greater than 110 mm Hg. That vacuum pressure results in the collapse of the capillaries that feed the apex tissue during a large percentage if not the entire cardiac cycle.

Perfusion Comparison

Further experiments were carried out to determine the perfusion rate in tissue captured by the inventive cup and the Xpose cup. The objective of the experiment was to examine and compare the alteration in flow within the transmyocardial space about the apical region of the heart during the application of the inventive device and the Xpose device for both the supine and vertical position of the heart. The myocardial blood flow was measured prior to and during the application of each the apical suction devices using myocardial blush analysis.

Angiography with GE OEC 9800 was performed in eight pigs with the inventive device or Xpose device applied in conformance with an alternating testing sequence shown in Table I. The first half of the evaluation was performed with the heart positioned in near anatomical orientation in a pericardial cradle. The second phase of this evaluation examined the devices with the apex of the heart repositioned or suspended in a vertical orientation. Angiography was performed of the apex region prior to and during the application of each vacuum suction device and blood flow was assessed through silhouette of the vasculature, myocardial blush response and timed rate of washout of the contrast agent.

The application of an external suction device to the epicardial surface for heart retraction may result in compression of the underlying vasculature if the transluminal pressure becomes less then zero. Reduction in the perfusion of the underlying musculature can result in cardiac rhythm disturbances, ischemia, and decreased cardiac function.

Alteration in myocardial blood flow in the apical region was assessed from the degree of myocardial blush prior to and during the application of each of the apical suction devices. The myocardial blush was measured with the cup positioned horizontally for the first four pigs tested and vertically for the last four pigs. Myocardial blush grades were defined as follows: 0, no myocardial blush or contrast density; 1, minimal myocardial blush or contrast density; 2, moderate myocardial blush or contrast density but less than that obtained during angiography of a contralateral or ipsilateral non-infarct-related coronary artery; and 3, normal myocardial blush or contrast density, comparable with that obtained during angiography of a contralateral or ipsilateral non-infarct-related coronary artery.

TABLE I
Blush Results

| | | Myocardial Blush |
|---|---|---|
| Animal | Device | Grade |
| 1 | inventive device | 3 |
| 1 | Control | 3 |
| 1 | Xpose device | 0 |
| 2 | Xpose device | 0 |
| 2 | control | 3 |
| 2 | inventive device | 3 |
| 3 | inventive device | 3 |
| 3 | control | 3 |
| 3 | Xpose device | 0 |
| 4 | Xpose device | 0 |
| 4 | control | 3 |
| 4 | inventive device | 3 |
| 5 | inventive device | 3 |
| 5 | control | 3 |
| 5 | Xpose device | 0 |
| 6 | Xpose device | 0 |
| 6 | control | 3 |
| 6 | inventive device | 3 |
| 7 | inventive device | 3 |
| 7 | control | 3 |
| 7 | Xpose device | 1 |
| 8 | Xpose device | 3 |
| 8 | control | 3 |
| 8 | inventive device | 3 |

Referring to Table I above, the inventive device revealed good filling and washout of the contrast agent in both the horizontal and vertical positions. The Xpose device impaired flow in each of the eight applications. In at least three applications the Xpose device deformed the apex of the heart into the shape of a “nipple”, and the apex of the heart was hypokinetic/akinetic following at least one application. As demonstrated by these evaluations, the inventive device preserved coronary circulation beneath the cup, while the Xpose device compromised the interfered with coronary circulation beneath the cup in all applications.

As a result of the compliant cup wall 231 of the inventive device, the surface area of captured tissue exposed to a high vacuum is reduced. Even at the lower vacuum pressures, the vacuum force necessary to reposition the heart is maintained. Thus, the user may position the cup to a position other than the heart's native position by using the vacuum force provided by the cup to resist the gravitational force of the heart on the cup.

Referring now to FIG. 9, there is shown an alternative implementation of the suction cup portion 112 of the suction cup device 110 of the present invention. In the alternative implementation illustrated in FIG. 9, an elastic mesh 164 is disposed in the cavity 132 proximate the lower rim 158. The elastic mesh 164 material is preferably Merselene or Prolene or other elastic type material. Prolene and Merselene fiber mesh are non-absorbable knitted products that are flexible and compliant yet afford excellent strength, durability, and surgical adaptability. The elastic mesh 164 can be disposed on the suction cup or attached thereto, such as by bonding, heat staking, or by an o-ring support. If bonded, a bonding material such as lactite is preferably used to attach the elastic mesh 164 directly on the inner surface 160. If heat staked, the suction cup portion 112 material is melted onto a surface of the elastic mesh 164. Of course, in such a bond, the melting point for the suction cup portion 112 material is lower then the melting point for the elastic mesh 164 material. If supported with an o-ring (not shown), the o-ring of an elastic material is overmolded on the circumferential edge of the elastic mesh 164 and the mesh/o-ring combination is inserted into the cavity 132 without bonding, preferably at the junction between the wall 130 and the lower rim 158. The o-ring (not shown) retains the elastic mesh 164 in the cavity 132 and behind the lower rim 158 and also allows for added flexibility of the mesh.

FIG. 10 illustrates the elastic mesh 164 prior to insertion in the cavity 132 of the suction cup portion 112. As shown in FIG. 9, the elastic mesh 164 is preferably inserted having a convex shape which engages the tissue or organ that is being supported. To facilitate the manipulation of the elastic mesh 164 into the convex shape, the elastic mesh 164 preferably has a plurality of triangular cut-outs 166 formed at equal spacings along its circumference. Those skilled in the art will appreciate that the elastic mesh 164 supports the tissue or organ as the suction retains the tissue or organ in position. The elastic mesh 164 also prevents tissue damage and minimizes the possibility of vacuum line clogging.

Referring now to FIGS. 11 and 12, there is shown another alternative embodiment of the suction cup portion 112 of the suction cup 110 of the present invention. In the alternative version illustrated in FIGS. 11 and 12, a closed-cell foam 168 is disposed on the lower rim 158 to engage the organ or tissue to be supported. The closed cell foam 168 is preferably cylindrical and having an opening 170 corresponding with the opening 159 formed by the lower rim 158. The closed cell foam 168 is preferably a hydrophobic closed cell foam. The close cell foam 168 can be attached to the lower rim 158 by any means known in the art, such as by adhering with an epoxy, a solvent weld, or heat weld.

In a pig study, the hydrophobic close cell foam 168 on the lower rim 158 showed the best tissue/organ attachment compared to hydrophilic close cell foam, rubber, and silicone. In addition, the hydrophobic close cell foam 168 induced the least amount of tissue injury (ecchymosis) and conformed best to cardiac apical and lateral regions. The pig study also showed that the compliant characteristic of the close cell foam 168 was critical in conformability. Thus, the hydrophobic closed cell foam 168 on the lower rim 158 allows cardiac contraction while maintaining vacuum seal, secured attachment without tissue injury, and conforms to the apical and lateral attachment positions of the heart.

Although discussed separately, the circumferential and radial channels 162a, 162b, the elastic mesh 164, and the closed cell foam 168 can be used in any combination in the suction cup 112, including all such features.

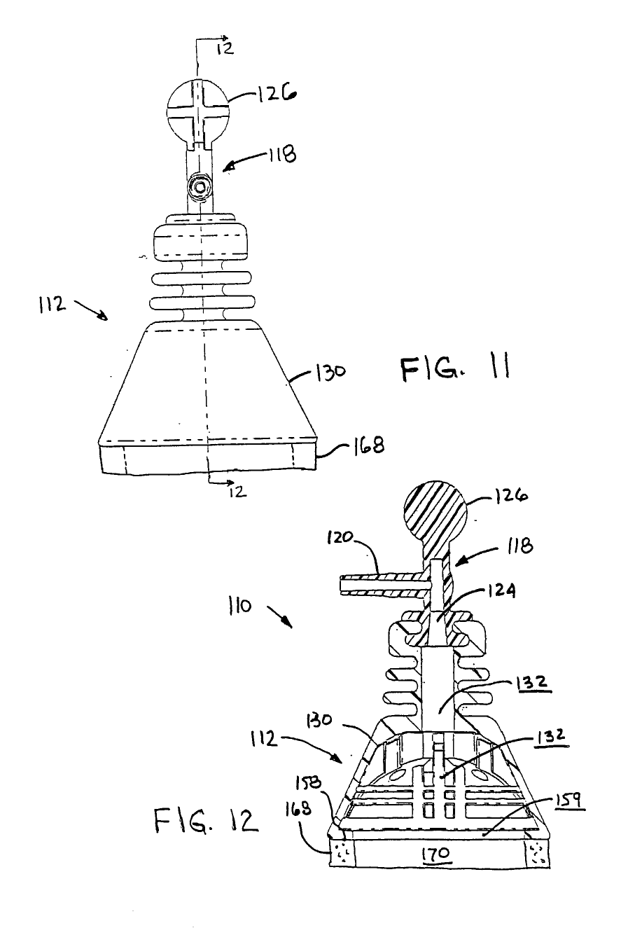

Referring now to FIGS. 13 and 14, there is shown a preferred mounting means 107 for slidable attachment to the side rail 102. The mounting means 107 is shown in phantom lines in FIG. 13 to clearly show its relationship with the side rail 102. In addition to being slidable along the side rail 102 into a desired position, the mounting means 107 must also lock into the desired position to prevent further movement of the suction device 106 during the surgical procedure being performed. The suction device 106 can have any one of the typical mounting means known in the art, such as the screw down mount 107 shown in FIG. 2. The screw down mount 107 typically has a knob 172, a base 174, and a key (not shown). The knob 172 threadingly engages the key through the base 174 such that when the knob 172 is tightened, the key urges against a slot (not shown) on the underside of the side rail 102 to lock the suction device 106 in the desired position.

Referring back to FIGS. 13 and 14, a preferred mounting means 107 is shown. As illustrated in FIG. 13, the side rail 102 has at least one edge 176 (referred to hereinafter as a “first edge”), which is non-linear. Preferably, the side rail has a second edge 178 that mimics the curve of the first edge 176. The non-linearity of the first and second edges 176, 178 can be a simple radius (r) as is illustrated in FIG. 13, or it can be have a plurality of curved and/or straight segments. The first and second edges 176, 178 preferably are cantilevered from a base 180 of the side rail 102 to form a “t” cross-section. The mounting means 107 preferably has a body 182 having a channel 184 substantially corresponding to the “t” cross-section of the side rail 102. The channel 184 has a linear width (w) such that it can be wiggled (applying a back and forth motion along direction ±A while maintaining a force (F) in the +A direction to move the body 182 in the +A direction) along the curved edges 176, 178 into a desired position and will stay locked in the desired position absent further wiggling of the body 182. To facilitate the wiggling of the body 182, a tab 186 is provided which protrudes from the body 182, preferably in a direction away from the opening in the body so as not to obstruct a surgeons view or access into the body.

While the side rail 102 is shown by way of example as having non-linear edges 176, 178 and the body 182 of the mounting means 107 is shown having a linear channel 184 width, those skilled in the art will appreciate that an opposite configuration will function in the same manner. That is, a side rail 102 having straight edges (not shown) and a mounting means 107 having a body with a curved channel (not shown) will operate similarly to the configuration described above in that the mounting means 107 can be wiggled into a desired position and would remain in the desired position absent further wiggling. Furthermore, while the side rail 102 is described by way of example as having cantilevered edges 176, 178, and the body 182 of the mounting means 107 is described as having a corresponding channel 184, those skilled in the art will also appreciate that the body 182 of the mounting means 107 can have cantilevered edges (not shown) and the side rail 102 can have a corresponding channel (not shown). Such an alterative configuration would also have the same intended function as the configurations described above in that the body 182 can be wiggled into a desired position and remain there absent further wiggling. Those skilled in the art will appreciate that the preferred mounting means 107, in any of the configurations discussed above, provides several advantages over the screw down type of mounting means of the prior art. For example, the mounting means 107 described above is less complicated and more economical since it has no moving parts. Furthermore, the preferred mounting means 107 described above requires a single hand for manipulation thereof, thus, eliminating the need for an assistant for placement and locking of the suction device 106 into a desired position.

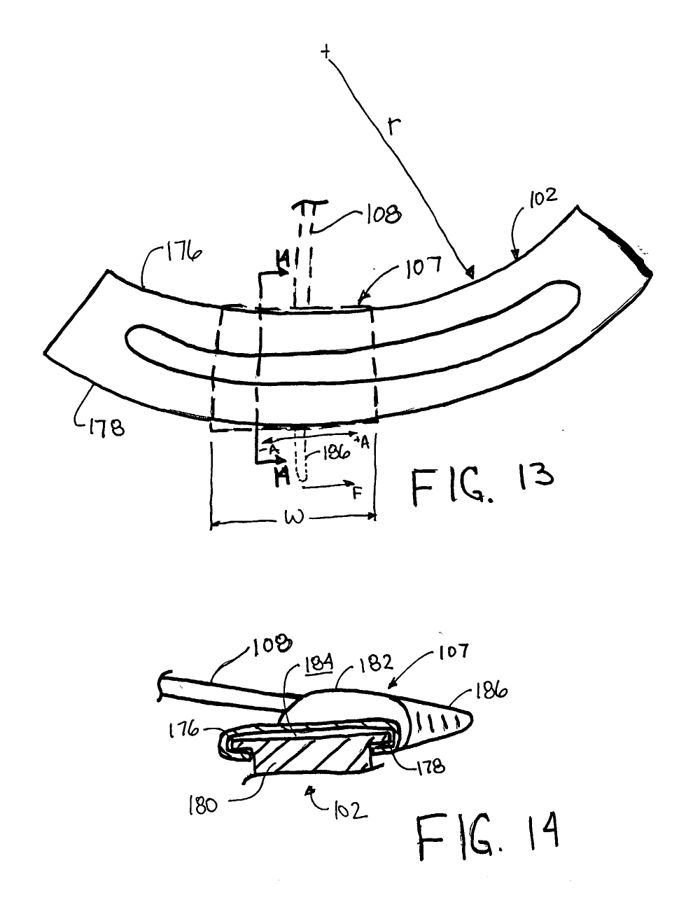

Referring now to FIGS. 15a, 15b, 16a, 16b, 17a, and 17b, there are shown cross-sectional views of three variations of a mounting means 107. Each of the mounting means 107 has a body 182 having a channel 184 formed therein. The channel 184 may have a straight or curved width and may be utilized with the preferred mounting means as discussed above with regard to FIGS. 13 and 14, or the channel 184 may be used with other mounting means known in the art, such as a screw down type. Each of the channels 184 depicted in the mounting means 107 of FIGS. 15a, 15b, 16a, 16b, 17a, and 17b, engage a side rail 102 having a base 180 with cantilevered edges 176, 178.

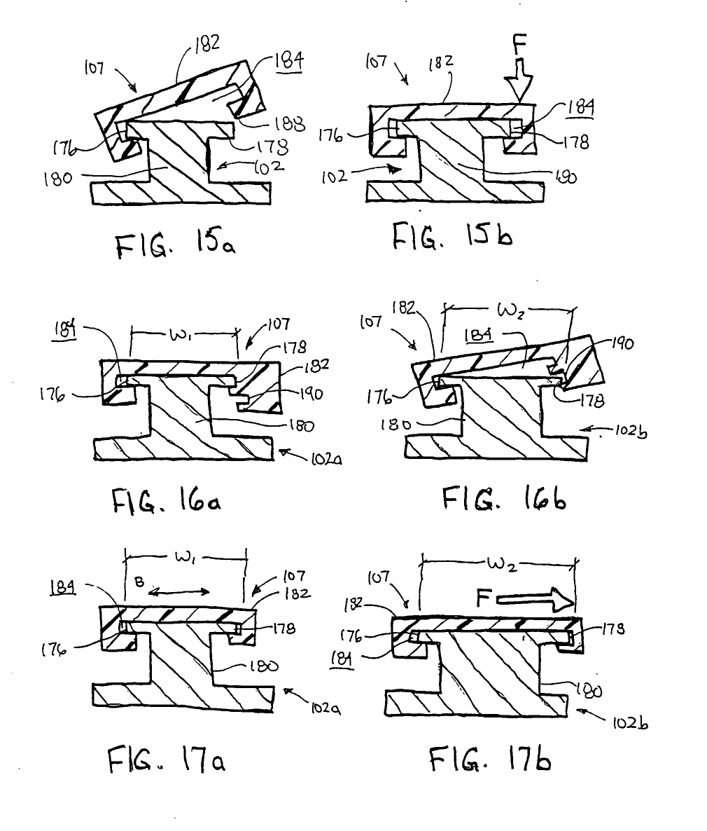

Referring specifically to FIGS. 15a and 15b, a first variation of the mounting means 107 is shown in which a force F is required in the direction of arrow F to secure the mounting means 107 on the side rail 102. FIG. 15a shows a slight interference between a portion 188 of the body 182 of the mounting means 107 and one of the edges (shown as the second edge 178) of the base 180 of the side rail 102. Such interference exists when the other of the edges (shown as the first edge 176) is placed in a corresponding portion of the channel 184 and the interference portion 188 rests on the other edge 178. A downward force F is applied to the body 182 in the vicinity of the interference portion 188 to force the second edge 178 into a corresponding portion of the channel 184 as shown in FIG. 15b. This type of fit between mating parts is commonly referred to as a “snap” fit. To facilitate the snap fit between the body 182 of the mounting means 107 and the base 180 of the side rail 102, at least a portion of the body 182 is preferably fabricated from a material having enough elasticity to plastically deform under the applied force F. Preferably, at least the body 182 of the mounting means 107 corresponding to the channel 184 is made from a thermoplastic. Referring now specifically to FIGS. 16a and 16b, there is shown a second variation of the mounting means 107. In the second variation, the body 182 of the mounting means 107 has a channel 184 with at least one extra slotted portion 190 for accommodating side rails 102 of varying widths w1, w2. FIG. 16a shows a side rail 102a having a first width w1 between the first and second edges 176, 178. The body 182 of the mounting means 107 is shown secured on the base 180 of the side rail 102a such that the first and second edges 176, 178 are disposed in the channel 184 and the mounting means 107 is substantially coplanar with the side rail 102a. FIG. 16b shows a side rail 102b having a second width w2, greater than the first width w1. However, the same mounting means 107 can accommodate either of the side rails 102a, 102b. As shown in FIG. 16b, one of the edges (shown as the first edge 176) is disposed in a corresponding portion of the channel 184 as discussed above. However, the other of the edges (shown as the second edge 178) is disposed in the extra slotted portion 190. Although in this configuration, the body 182 of the mounting means 107 is slightly inclined with respect to the side rail 102b, the operation of the suction device 106 is not altered due to the articulation of the arm 108 and the pivoting of the suction cup 110 relative to the arm 108 provided by the pivot 109.

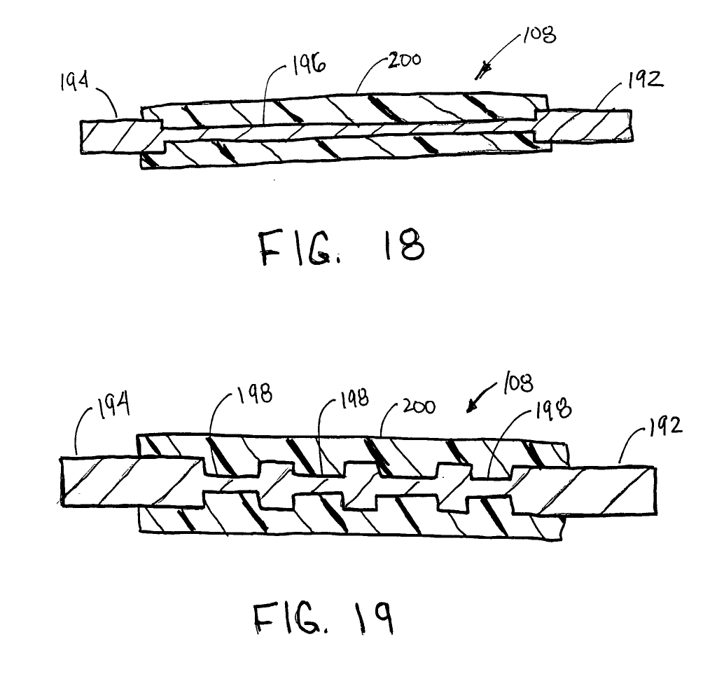

Referring now specifically to FIGS. 17a and 17b, there is shown a third version of the mounting means 107, which like the second version shown in FIGS. 16a and 16b, can accommodate side rails 102 of different widths w1, w2. FIG. 17a shows the body 182 of the mounting means 107 secured on the side rail 102a. Specifically, the first and second edges 176, 178 are disposed in corresponding portions of the channel 184. The body 182 of the mounting means, or at least the portion of the body 182 corresponding to the channel 184 is fabricated from a stretchable material, such as an elastomer, such that it can be stretched in the direction of arrow B. FIG. 17a shows the body 182 in a relaxed (unstretched) state secured on a side rail 102a having a width w1 between the first and second edges 176, 178. FIG. 17b shows the same body 182 stretched in direction B by the application of a force F to fit over a side rail 102b having a width w2, greater than width w1. Those skilled in the art will appreciate that unlike the second version shown in FIGS. 16a and 16b, the third version of the mounting means 107 can accommodate side rails 102 having a range of widths.

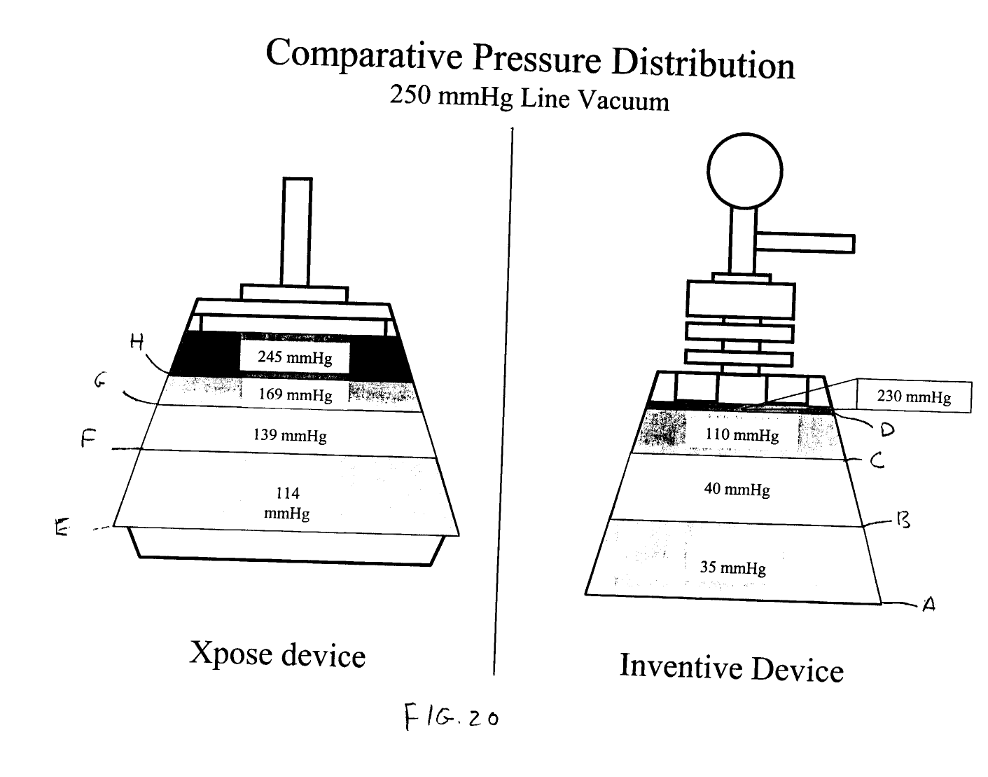

Referring now to FIGS. 18 and 19, there is illustrated the arm 108 of the suction device 106. The arm 108 is shown in FIGS. 18 and 19 apart from its mating portions of the suction device 106. A first end of the arm 192 is fixed in the mounting means, preferably, by a force fit, braze, or other means known in the art. A second end 194 of the arm 194 is disposed in the distal adaptor 128, preferably in a rotating fashion. The arm 108 is preferably of a unitary construction having a central undercut portion 196, or alternatively, a series of undercut portions 198 as shown in FIG. 19. The arm 108 is fabricated from a malleable material which can be deformed into a desired shape yet still be resilient enough to remain in such deformed position to support an organ or tissue cantilevered at the suction cup 110. Preferably, the malleable material is a type 304 annealed stainless steel.

The arm 108 can be used in either a straight configuration, as shown in FIGS. 3a and 3b, or in a curved configuration, as shown in FIGS. 1 and 2. A cushion material 200 is preferably disposed around all portions of the arm 108 except the first and second ends 192, 194. The cushion material 200 can be prefabricated and applied on the arm 108 or molded directly onto the arm 108. The cushion material can be any flexible material, such as c-flex, which aids in the resiliency of the arm. Those skilled in the art will appreciate that the arm 108 of the present invention has many advantages over the arms of the prior art, including, simplicity of design (contains no moving parts), ease of operation (does not have to be actuated into and out of a locked position), and low profile (does not encumber the surgeons view or access to the surgical site.

While there has been shown and described what are considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A suction device for applying vacuum to a tissue surface, the suction device comprising a suction cup portion having a vacuum inlet, the suction cup portion having a wall defining a cavity in fluid communication with the vacuum inlet and an engagement surface for engaging the tissue surface, the suction cup portion being formed of a material having a durometer of less than 40 Shore A.

2. The suction device of claim 1, wherein the material has a durometer between 20 and 40 Shore A.

3. The suction device of claim 2, wherein the material has a durometer of about 30 Shore A.

4. The suction device of claim 1, wherein the thickness of the wall is between 0.054 and 0.066 inches.

5. A suction device for applying vacuum to a tissue surface, the suction device comprising a suction cup portion having a vacuum inlet, the suction cup portion having a wall defining a cavity in fluid communication with the vacuum inlet and an engagement surface for engaging the tissue surface, the suction cup portion being configured to have a controlled leak rate of approximately between 3 and 5 mm Hg/minute.

6. The suction device of claim 5, wherein the suction cup portion is configured to have a controlled leak rate of approximately 4 mm Hg/minute.

7. A method of positioning a heart with a suction device, comprising the steps of:
providing a suction device comprising a suction cup portion having a vacuum inlet, the suction cup portion having a wall defining a cavity in fluid communication with the vacuum inlet and an engagement surface, the suction cup portion being formed of a material having a durometer of less than 40 Shore A;
contacting an apex of the heart with the engagement surface of the cup portion;
supplying a vacuum to the vacuum inlet to capture heart tissue within the suction device;
conforming a substantial portion of the wall of the suction device to a portion of the apex of the heart; and
repositioning the heart using the suction device while permitting substantial perfusion of the heart tissue captured within the suction device.

8. The method of claim 7, wherein the suction device is configured to permit a controlled leak rate and the supplying step comprises permitting a controlled leak rate of the vacuum supplied to the vacuum inlet via the engagement surface of the suction device of between 3 and 5 mm Hg/minute.
